(19) United States
(12) Patent Application Publication
(10) Pub. No.: US 2006/0155186 A1
(43) Pub. Date: Jul. 13, 2006

(54) Bone health assessment using spatial-frequency analysis

(76) Inventor: Timothy W. James, Goleta, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/064,381

(22) Filed: Feb. 23, 2005

Related U.S. Application Data
(60) Provisional application No. 60/593,417, filed on Jan. 12, 2005.
(60) Provisional application No. 60/593,871, filed on Feb. 19, 2005.

(51) Int. Cl. A61B 5/05

(52) U.S. Cl. 600/410

(57) ABSTRACT

Bone health assessment using spatial-frequency analysis for assessing the health of trabecular bone by acquiring k-space data for the relevant spatial frequencies and direction vectors indicative of bone health. This does not require that the k-space data be taken with the bone held motionless for the duration of the analysis. The preferred method of acquiring this data is to use a magnetic resonance device with the ability to measure k-space values for the appropriate spatial frequencies and direction vectors, a requirement which greatly reduces the required complexity and cost of the device over conventional MRI equipment. Magnetic resonance is particularly well suited to this, as bone gives very low signal and marrow (which fills the spaces between the lattice of trabecular bone) gives high signals hence providing good contrast. Various exemplary data acquisition and analysis techniques are disclosed.

### CROSS-REFERENCE TO RELATED APPLICATIONS

This application claims the benefit of U.S. Provisional Patent Application No. 60/593,417 filed Jan. 12, 2005 and U.S. Provisional Patent Application No. 60/593,871 filed Feb. 19, 2005.

### BACKGROUND OF THE INVENTION

1. Field of the Invention

The present invention relates to the field of diagnostic assessment of bone strength in patients at risk of or suffering from osteoporosis and other conditions which degrade the trabecular structure of cancellous bone.

2. Prior Art

The trabecular architecture is both highly sensitive to metabolic changes in bone (relative to the more dense outer shell of cortical bone) and a major contributor to the overall strength of a bone. Hence it is an appropriate surrogate marker for tracking disease and treatment.

The Impact of Bone Disease Diseases of the skeletal system, including osteoporosis and other less common conditions, are a major threat to the health of the elderly, particularly women. The significance of bone disease is evident from the 2004 Surgeon General's report, “Bone Health and Osteoporosis,” and from the declaration of 2002-2011 as the Decade of the Bone and Joint, by President George W. Bush. More than 10 million Americans over age 50 suffer from osteoporosis (the weakening of the skeletal system as a result of loss of bone mass), and an additional 34 million are at risk. More than 1.5 million fractures occur each year as a result of osteoporosis, with direct costs of care of approximately $15 billion, and billions more in costs associated with loss of productivity and the three-fold increase in risk of mortality associated with fractures. The continuing aging of the population will cause the number of fractures and the associated economic and societal impact to more than double by 2020, with at least 50% of the population over the age of 50 suffering from, or at risk of, osteoporosis.

Diagnosis and Treatment of Osteoporosis The cycle of bone production goes through a number of stages, typically peaking in the early twenties and declining gradually thereafter. In middle age, and particularly in post-menopausal women, the net production of bone can become negative, and the trabecular bone, the structure of rods and plates that supports the outer shell of cortical bone, becomes thinner and weaker. This degradation is illustrated by a comparison of FIGS. 1 and 2, which show excised sections through, respectively, healthy bone and osteoporotic bone. The calcified bone is bright in these images and the regions which would have been filled with marrow in living tissue are dark. The loss of bone strength that results from the thinned and more porous bone structure in osteoporotic bone increases the risk of fracture in vulnerable regions such as the hip and spine. Although the hip and spine exhibit most of these fractures, they are more difficult to image than the calcaneous (heel bone) and distal radius. Since osteoporosis is a systemic metabolic disease, and the weight-bearing bones are good indicators of the disease state, images of either of these bones are indicative of the progression of the disease in the patient's skeletal system as a whole. The calcaneous is a particularly good bone for assessing trabecular architecture, as it is a weight-bearing bone and relatively accessible for imaging using an MRI (magnetic resonance imager or magnetic resonance imaging).

Osteoporosis is not an inevitable consequence of aging. Proper lifestyle choices, including smoking cessation, moderate exercise, and adequate doses of calcium and vitamin D, can reduce bone loss and fracture risk. Several drugs are also available for the treatment of osteoporosis. Bisphosphonates, including Fosamax™ and Actonel™, are oral agents that reduce the resorption of bone. Teriparatide, marketed under the name Forteo™, is an anabolic hormone extract that stimulates bone growth but must be administered by daily injection. Other forms of hormone therapy also stimulate development of bone but carry significant risk of side effects as shown in recent clinical trials.

Proper therapy requires timely and accurate diagnosis. The current standard in diagnosis of osteoporosis is measurement of bone mineral density (BMD) by dual energy x-ray absorptiometry (DEXA). Recent studies have indicated that DEXA is underutilized, with less than 25% of the at-risk population receiving BMD testing, due partially to the cost of DEXA but primarily to lack of awareness. Of much greater concern is the fact that physicians have begun to question the clinical relevance of DEXA, based on emerging evidence that DEXA measurements do not properly predict fracture risk and are particularly inadequate in assessing the effectiveness of therapy.

As a result of these concerns, a number of other imaging modalities, including quantitative computed tomography, ultrasound, and magnetic resonance imaging are being explored as alternatives to DEXA. The resistance of bone to fracture depends, as is the case for most materials, not just on density but also on the structure of the bone, including the relative fractions of, and the thickness and orientation of, trabecular rods and plates. MRI, which is inherently a three-dimensional technique, is well suited to the determination of the structural details that determine fracture resistance.

The MRI techniques currently being investigated for diagnosis of osteoporosis require the acquisition of extremely high-resolution images, as well as requiring a number of image processing operations. FIG. 3 is an MR image obtained from an excised bone sample using a 7 Tesla high field MRI device. In FIG. 3, as in living tissue, MR images have high signal in the marrow and low signal from the hard calcified bone. Images of living bone can be acquired in a high-field MRI system using specialized coils, and lengthy exam times. Careful patient positioning and stabilization are also required. These high-field systems cost around $2 million and need to be housed in carefully controlled environments overseen by radiology specialists. The invention reported here enables devices that can be housed in a typical doctor's office and which cost less than $200,000.

Magnetic Resonance (MR) in some ways is particularly well suited to measuring living bone, as hard-bone (i.e., the calcified structure of the trabeculae and cortical bone) gives very low signal, while marrow (which fills the spaces between the trabecular lattice) gives high signals, hence providing good contrast and good signal to noise. But the high cost of high-field systems, and the need for long acquisition times in order to resolve fine structures combined with the requirement that the patient (imaged body part) not move during acquisition, yield a level of impracticality in the implementation of standard MRI for this purpose.

MRI is based on an extension of the mathematics of Fourier expansion which states that a one-dimensional repetitive waveform (e.g., a signal amplitude as a function of time or an intensity as a function of linear position) can be represented as the sum of a series of decreasing period (increasing frequency) sinusoidal waveforms with appropriate coefficients (k-values).

In MRI, the item (body part) to be imaged is a three-dimensional object. The basic concept of k-values in one dimension can be extended to two or three dimensions. Now, rather than a series of k-values, there is a two or three-dimensional matrix of k-values, each k-value representing a particular spatial frequency and direction in the sample.

In Fourier analysis, converting from the k-values to the desired waveform (amplitude vs. time for a time varying signal or image intensity vs. position for the MRI case) is accomplished by using a Fourier transform. The Fourier transform in simple terms is a well-known means to convert between the frequency domain and time domain (for time varying signals). For images, as in the MRI case, the Fourier transform is used to convert between the spatial-frequency domain (the series of sinusoidal waveforms and their coefficients, referred to as k-space) and the spatial arrangement of signal intensities for each of the imaged volumes (voxels). Similar to the case of time-varying signals, where the k-values are coefficients for the sinusoidal waveforms with given periods, the k-values in the MRI case are the coefficients for the sinusoidal waveforms with given wave lengths (where the wavelengths are inversely related to spatial frequencies, i.e., a long wavelength is a low spatial frequency).

MRI technology today uses a number of methods to acquire images. Virtually all rely on gathering the k-space coefficients and later Fourier transforming them into an image (or set of images as in a 3D acquisition). In the simplest abstraction, this is accomplished by placing the part to be imaged in a strong magnetic field and exciting the hydrogen nuclei in the sample by transmitting at the sample a pulsed radio-frequency electromagnetic signal tuned to the resonant frequency of the hydrogen nuclei. This pulse starts the nuclei resonating at their resonant frequency. Then, to obtain information about where in the sample the signal originates from, the spins of the excited hydrogen atoms are encoded with a combination of phase and frequency encodes corresponding to the desired k-space data being acquired on that excitation. (Here phase and frequency refer to the resonant frequency and phase of the hydrogen nuclei). This is accomplished by modulating the magnetic field spatially and temporally, so as to correspondingly spatially alter the resonant frequency of the nuclei and modulate their phase. A signal is received back then from the excited hydrogen nuclei of the sample, and the k-values are extracted from the signal. This process of excitation, encoding, and signal acquisition is repeated until an entire matrix of k-space values (properly selected to constitute a Fourier series) is acquired with sufficiently high spatial frequency to resolve the desired features in the sample. Finally, the matrix of k-values is Fourier transformed to produce an image or images. There are many variations and extensions of this theme in use in current technology MRI systems. One approach utilizes frequency encoding to localize signals to thin slices and phase encoding to generate the k-values for each of these 2D slices.

### BRIEF DESCRIPTION OF THE DRAWINGS

FIG. 1 is an image of a specimen of healthy trabecular bone showing a fine highly interconnected structure of trabeculae.

FIG. 2 is an image of a specimen of osteoporotic trabecular bone showing a significantly less fine and interconnected structure of trabeculae than in FIG. 1.

FIG. 3 is a single thin slice high resolution MR image showing the trabecular structure of a 15mm excised bone cube obtained with the use of a 7 Tesla MRI system.

FIG. 4 is a diagram illustrating a simple implementation of a magnetic resonance device for acquiring numerical k-values from a patients bone and comparing the measured values with known reference values or previous measurements on the same patient.

FIG. 5 is a plot illustrating acquiring k-values in multiple regions of K-space along the horizontal axis in a region near the origin (i.e., low k-values corresponding to low spatial frequencies, i.e., long spatial dimensions) and two regions at higher spatial frequencies corresponding to smaller dimensions.

FIG. 6 is a plot illustrating acquiring a number of k-values in a region encompassing a range of spatial frequencies and a range of directions spread over the angle phi centered on a principal anatomical direction.

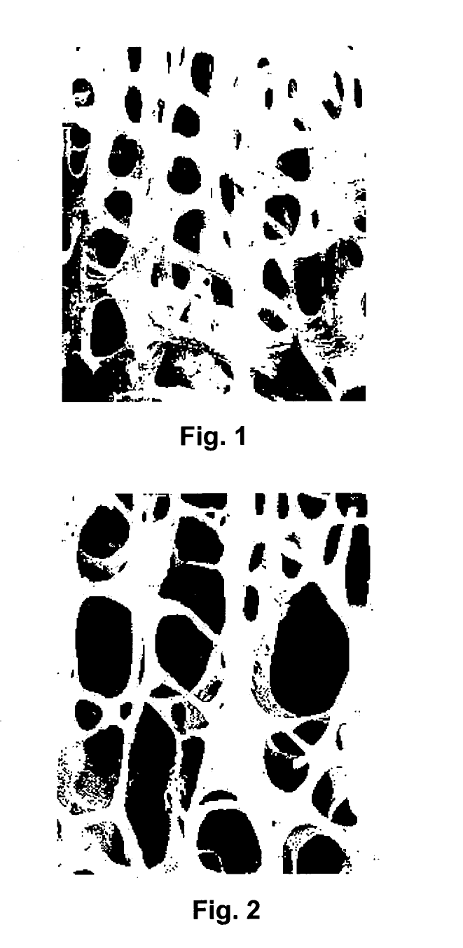

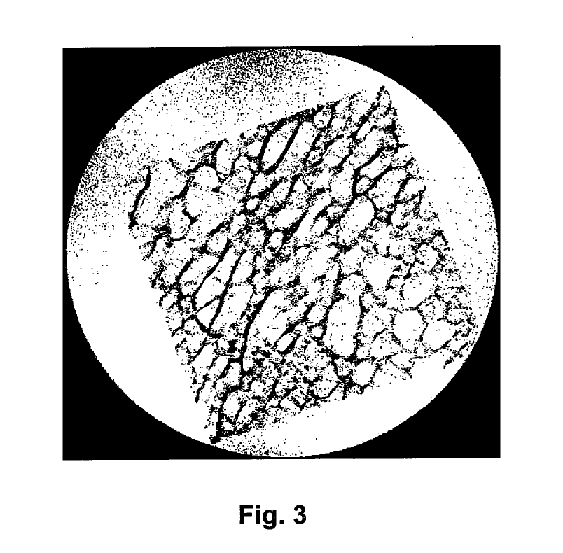

### DETAILED DESCRIPTION OF THE PREFERRED EMBODIMENTS

The present invention is a far simpler and more elegant solution to diagnosing osteoporosis by MR (magnetic resonance) than the prior art. The method is based on the fact that the acquisition of data using MR is performed in Fourier reciprocal space, or k-space. K-space data represents spatial frequencies, which correspond to spatial distances in real space, but in an inverse relationship—the shorter the distance the higher the k-values. Healthy trabecular bone exhibits a certain characteristic range of spatial frequencies, while osteoporotic bone exhibits a different characteristic range. Analytical comparison of the spectrum of k-space numerical values (spatial-frequency coefficients, or “k-values”) obtained by MR from a patient's bone, with data typical of healthy and osteoporotic trabeculae, respectively, will provide definitive characterization of the health of the patient's trabecular bone, which will then determine the risk of fracture and the need for therapy. This technique should be implementable in virtually any type of MR data acquisition device, including the MR data acquisition devices in conventional MRI equipment.

The preferred means for acquiring this data is to use an MR device with the ability to gather k-space values for the appropriate spatial frequencies and direction vectors. MR is particularly well suited to this, as bone gives very low signal, while marrow (which fills the spaces between the bone trabeculae) gives high signals, hence providing good contrast.

Bone is a three-dimensional structure. A large part of the strength of a bone is provided by the trabecular lattice structure in cancellous bone in the medulary portion of the bone. This lattice structure is very sensitive to bone metabolic disease and other factors (e.g., exercise). Bone loss in this lattice structure results in loss of the fine structure of interconnecting webs and rods with a resultant coarser and less interconnected, hence weaker, lattice.

The approach of this invention is to acquire k-space data for only the spatial frequencies and direction vectors relevant to determining and assessing the health (e.g., degree of osteoporosis) of trabecular bone structure and in determining changes in the trabecular structure. By use of this approach, an assessment of the health of trabecular bone can be made by taking data at a much smaller range of spatial frequencies (k-values) than is required in standard MRI imaging. Furthermore since this invention relies on analysis of a portion of the k-space spectrum rather than an image, the k-values can be acquired without regard to satisfying the strict requirements for k-values suitable for Fourier transforming into an image. The requirements for images require that all k-values be taken with the sample in precisely the same position (i.e., in the same spatial phase), and that the k-values precisely match the spatial frequencies of a particular Fourier series. Because in this case the numerical k-values, not an image, are the goal, this invention removes the need for keeping the bone completely immobile for long data acquisition times, and greatly simplifies the MR data acquisition device and its capability requirements, allowing use of much simpler and significantly less costly machines.

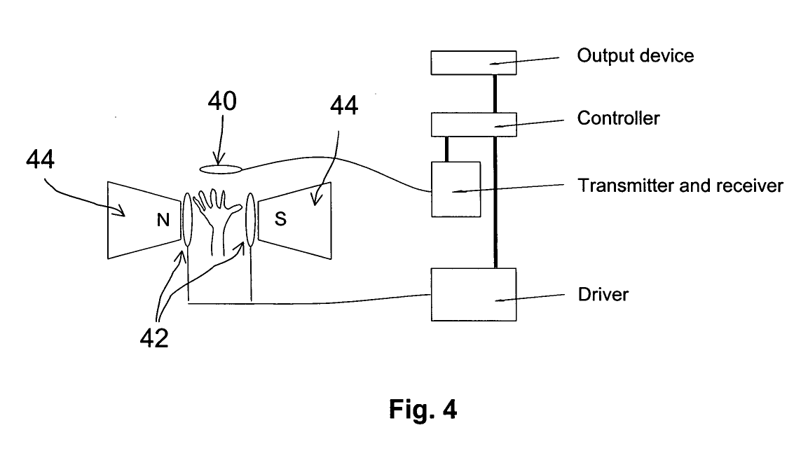

FIG. 4 illustrates a simple implementation of a magnetic resonance device for measuring numerical values of specific k-space spatial frequencies and directions for use in evaluating bone trabeculae. The system consists of a magnet 44 to generate a field in the region of the bone to be sampled (here a bone of the wrist), an antenna 40 coupled to a transmitter for transmitting to and exciting the hydrogen nuclei, a magnetic field modulator 42 connected to a driver for modulating the magnetic field spatially and temporally, an antenna and receiver to receive the MR signal consisting of a receiver and an antenna 40 which can be the same as used for transmit or a separate device, a controller connected to the transmitter, receiver, driver, and a user interface which includes an output device for calculating and reporting the results. The controller controls the excitation, encoding, and receive processes to gather the desired k-values from the specimen 41 and subsequently performs k-value extraction processes. Data analysis and report generation would be performed either by the controller or other conventional approaches.

There are many possible variations of this basic configuration. These include having multiple magnetic field modulators and drivers to encode in additional directions and having separate transmit and receive antennas.

Rather than require that the patient keep perfectly motionless, in a preferred embodiment of this invention, it is actually desirable to acquire k-value data for more than one position of the sample relative to the MR device. This could be accomplished by asking the patient to reposition one or more times during the data acquisition or by use of a mechanical device. The acquisition time at each position can be on the order of seconds, rather than the several minute scans required for conventional imaging, a huge improvement in practicality and patient comfort.

There are many possible ways to implement this invention. A simple implementation of this invention would be to use a device that would selectively acquire the k-values for a single spatial frequency (or would average a range of spatial frequencies) corresponding to healthy bone (e.g. in a range around a spatial frequency corresponding to about 0.5 mm in the heel bone—the exact spatial frequency analyzed depends in part on the direction in the bone being analyzed, the particular bone, and patient demographics). These k-values (usually represented as complex numbers) can be numerically compared with values typically found in normal and diseased bones representative of the patient's demographics, and with previous measurements of k-values taken on the same patient. The numerical comparison can be by comparing magnitudes of the k-values.

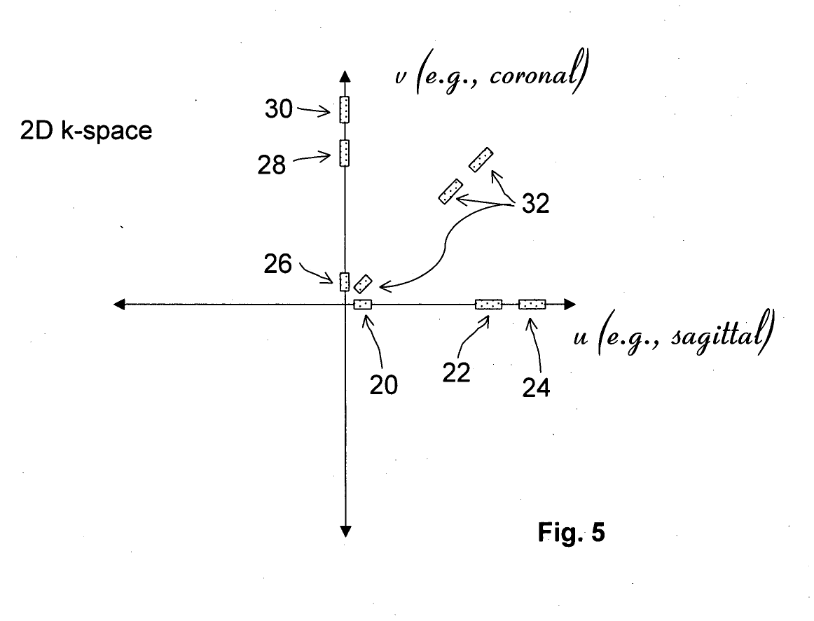

Alternate methods of comparison include averaging the k-values of one or more samples taken in a range of spatial frequencies around the range for healthy bone and comparing with the average of one or more samples in a range of spatial frequencies around that for unhealthy bone (e.g., 1.0 mm for the heel bone). This approach is diagrammatically illustrated in FIG. 5, which shows regions in k-space (here in the 2D case). A range of spatial frequencies around that of healthy bone in the sagittal direction 24 is shown on the u axis, also indicated is a second region 22 at lower spatial frequencies (longer characteristic dimensions representative of diseased bone). Also indicated in FIG. 5 is a region 20 of spatial frequencies in the sagittal direction with characteristic dimensions much longer than any of the trabecular bone structures is shown near the origin of the plot. The ratio of the measurements in regions 22 and 24 would be indicative of the amount of healthy bone present.

A second alternate method of comparison is to correct for probable offsets in the magnitude data which might arise due to differences between individual patients, disease state, or other time-varying effects that modify the marrow signal—one implementation would normalize the magnitude of one or more samples in the spatial frequency range corresponding to healthy bone 24 by also taking k-space data at spatial frequencies very much larger than that for healthy or diseased bone 20 (e.g., 10 mm). These long wavelength samples would be preferentially sensitive to the amount of marrow and to the marrow signal intensity itself as well as to the sensitivity (or gain) of the acquiring instrument. Normalizing the measurements in the spatial frequency range of healthy 24 and osteoporotic 22 bone by the long wavelength k-values 20 would make the measurement more sensitive to trabecular changes. Also indicated in FIG. 5 is the same set of measurements discussed above but in the coronal anatomical direction 26, 28, 30. 32 indicates making measurements at an intermediate angle to the primary anatomical directions.

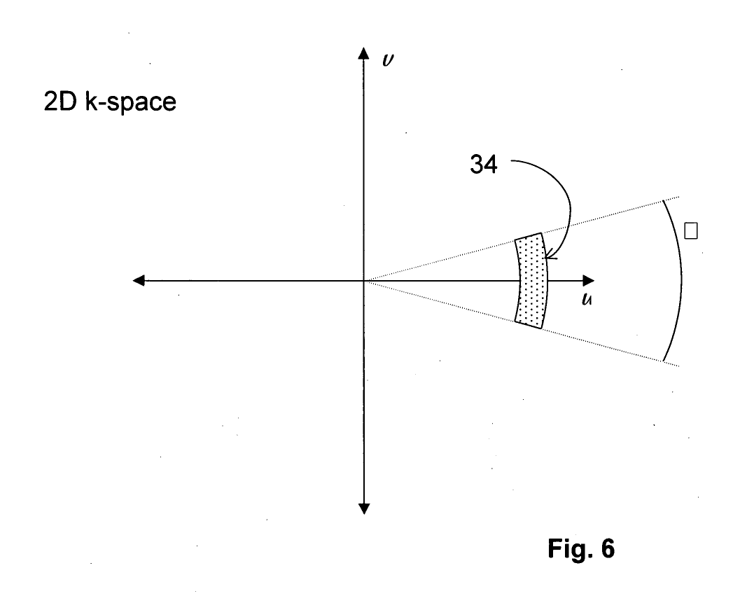

Because bone is anisotropic, it is anticipated that in order to get a representative measure of disease state, samples may be needed in more than one of the three anatomical directions (coronal, sagittal, and axial). It is also anticipated, because of the anisotropy and individual to individual variation, that averaging samples over a range of directions will give a more repeatable and representative measurement than a single direction. Alternatively an algorithm can be used to analyze the k-values as a function of direction and detect the representative value (e.g., maximum). This is illustrated in FIG. 6 (again in the 2D case), which illustrates the acquisition of k-values 34 over a small range of spatial frequencies and covering an angle of Ø centered around one of the principal anatomical directions. This sampling over a range of directions can be accomplished by rotating the patient's bone relative to the device, or by utilizing combinations of two encoding means 42. The maximum or dominant spatial frequency or frequencies may be determined various ways, such as by actually finding the frequency having the maximum k-value magnitude within a spatial frequency range spanning the primary spatial frequency range providing the best indicator of healthy and diseased bone, using a regression technique to fit a function to the data set and then analyzing the function for the characteristic value (e.g., maximum), or by summing the magnitudes of k-values for a plurality of successive spatial frequencies as a smoothing operation using a sliding window, and using the largest sum as an indicator of the respective spatial frequency or spatial frequency range. Of course whatever technique is used, the same would be applied to the k-values for healthy and diseased bone, and/or k-values previously obtained for the same spatial frequencies and same bone.

Further, it is also anticipated that acquiring multiple samples of the same k-value will enable determination of representative k-values with less data scatter. These multiple samples can be taken with the patient in the same position relative to the instrument, as well as with variations in patient position (translational rather than rotational). These variations in position can be contrived so that they are not equal to wavelength of the spatial frequency or an integral multiple or simple fraction of it. Samples taken in the same position would serve to reduce signal noise from the detection system and samples taken over multiple positions would help to reduce noise due to local variations in the sample itself. An alternate approach would be to take samples closely spaced around the same spatial frequency (as illustrated in FIG. 5 20 to 32. This would accomplish sampling various relations of major structures with the spatial phase of the k-value being acquired. (Again as this technique does not need to take specific k-values for subsequent transformation into an image there are no limitations to which samples might be taken).

A low cost MR data acquisition system might consist of a reduced functionality MR data acquisition system with a single phase-encoding gradient and single-frequency encoding gradient. If data was desired from other anatomical directions, the protocol could include repositioning the relative positions of the bone and the measuring apparatus.

The preferred embodiments of the invention are based on there being sufficient information in an appropriate subset of the entire 3-dimensional spatial frequency matrix (k-space matrix) to evaluate the lattice for its contribution to bone strength. This subset would include the appropriate spatial-frequencies (representative of the healthy fine lattice-structure) and appropriate anatomical directions (e.g. longitudinal to the bone and the two orthogonal directions).

Because the trabeculae are a continuous phase (i.e., there are not islands or small bits of bone floating in a sea of marrow) it is intuitively apparent that if a structure has a high value for spatial frequencies in the appropriate (healthy) range in all three orthogonal directions, that the lattice is fine and highly interconnected. The morphology of bone may also ensure that if there is a high value of the appropriate k-values (normalized or otherwise averaged over ranges of small ranges of anatomical directions) in two orthogonal directions, that this also ensures a highly-interconnected, healthy trabecular structure.

Thus, given a k-space data set, one can analyze it directly for its spatial frequency content (spectrum). By comparing the spatial frequency spectrum of the item (in this case, trabecular bone) being studied to that obtained from healthy trabecular bone, an assessment of the state of health of a person's bone structure can be made. Similar comparisons of the measured spectrum of k-values can be made over a period of time, to assess variations in a patient's bone structure over time. By tracking changes over time, an assessment of the efficacy of ongoing therapies can be made.

Accordingly, one aspect of this invention is to provide a method (or an implementation of a means using the method), which enables the practical use of MR data acquisition to assess changes in the trabecular structure of cancellous bone noninvasively. In particular, this invention eliminates the need for long data acquisition times, expensive MRI equipment, and precise, motionless positioning of the patient's anatomy, things which would otherwise be required to generate an image of the trabecular structure with sufficient detail to allow determining and tracking changes in its structure. The advantages of this invention over the prior art using MRI, as well as over current clinical practice using DEXA, are that it enables a simple, significantly-lower-cost magnetic resonance-based device (in contrast to DEXA, MR does not use ionizing radiation) to acquire the representative k-space numerical values to assess and track changes in the trabecular structure of cancellous bone.

This invention could be applied to data acquired by most any current MRI imager, though now the MR data acquisition system can be programmed to only acquire the desired sub-set of k-values, hence, significantly reducing the required acquisition time (from on the order of ten minutes or more in conventional practice down to seconds by use of this invention). The invention can be implemented as a software program for analyzing the data, or it can be implemented in a dedicated system with fewer components than are necessary in current MRI systems (e.g., a single phase-encode gradient rather than multiple ones).

Although the invention has been described with respect to specific preferred embodiments, many variations and modifications may become apparent to those skilled in the art. It is therefore the intention that the appended claims be interpreted as broadly as possible in view of the prior art to include all such variations.

What is claimed is:

1. A method of assessing the health of trabecular bone comprising obtaining k-values representing specific spatial frequencies and direction in the trabecular bone, and comparing those k-values with k-values from the same spatial frequencies and direction in bones with known degrees of disease.

2. The method of claim 1 where the k-values are obtained using magnetic resonance.

3. The method of claim 1 wherein k-values are obtained in multiple directions in the trabecular bone.

4. The method of claim 1 where the spatial frequencies are chosen to overlap the characteristic spatial frequency of healthy trabeculae in the type of bone being assessed.

5. The method of claim 4 where the spatial frequencies are also chosen to overlap the characteristic spatial frequency of diseased trabeculae in the type of bone being assessed.

6. The method of claim 5 where the ratio of the k-values obtained by claim 4 and in claim 5 are used as a measure of bone quality.

7. The method of claim 6 wherein the ratio is the ratio of the magnitudes of the k-values.

8. The method of claim 1 wherein the trabecular bone is moved and k-values representing the same spatial frequencies and direction in the trabecular bone are obtained and averaged with the k-values obtained before the movement.

9. The method of claim 8 wherein the amount of movement is not coherent with the spatial frequencies.

10. The method of claim 9 wherein the magnitude of the k-values are averaged.

11. The method of claim 8 wherein the trabecular bone is rotated about a principal axis and k-values representing the same specific spatial frequencies and direction in the trabecular bone are obtained and averaged with the k-values obtained before the rotation.

12. The method of claim 11 wherein the magnitude of the k-values are averaged.

13. The method of claim 1 wherein the k-values obtained are compared with k-values from the same spatial frequencies and direction in bones with known degrees of disease in a person of the same or similar demographics.

14. The method of claim 1 further comprised of obtaining multiple k-values for the same spatial frequency.

15. The method of claim 14 wherein the magnitudes of the multiple k-values are averaged.

16. The method of claim 1 wherein the spatial frequencies are closely spaced.

17. The method of claim 1 further comprising obtaining k-values representing long wavelength spatial frequencies and normalizing the k-values to be compared with k-values from the same spatial frequencies and direction in bones with known degrees of disease before the comparison.

18. The method of claim 17 wherein the k-values from the same spatial frequencies and direction in bones with known degrees of disease are normalized using k-values representing the same long wavelength spatial frequencies for each bone with the respective known degree of disease.

19. The method of claim 1 wherein the k-values obtained are also compared with k-values from the same spatial frequencies and direction in the patient as previously obtained.

20. The method of claim 1 further comprising determining dominant spatial frequencies and comparing the dominant spatial frequencies.

21. The method of claim 20 wherein the dominant frequencies are determined by determining the frequencies of the k-values having a maximum magnitude.

22. The method of claim 20 wherein the dominant frequencies are determined by determining the sum of the magnitudes of k-values for a predetermined number of successive spatial frequencies.

23. A method of assessing the health of trabecular bone comprising obtaining k-values representing specific spatial frequencies and direction in the trabecular bone, and comparing those k-values with k-values from the same spatial frequencies and direction in the patient as previously obtained.

24. The method of claim 23 where the k-values are obtained using magnetic resonance.

25. A method of assessing the health of trabecular bone comprising obtaining k-values representing specific spatial frequencies and direction in the trabecular bone using magnetic resonance, and comparing those k-values with k-values from the same spatial frequencies and direction in bones with known degrees of disease of persons of similar demographics.

26. The method of claim 25 wherein k-values are obtained in multiple directions in the trabecular bone.

27. The method of claim 25 where the spatial frequencies are chosen to overlap the characteristic spatial frequency of healthy trabeculae in the type of bone being assessed.

28. The method of claim 27 where the spatial frequencies are also chosen to overlap the characteristic spatial frequency of diseased trabeculae in the type of bone being assessed.

29. The method of claim 25 wherein the trabecular bone is moved and k-values representing the same spatial frequencies and direction in the trabecular bone are obtained and averaged with the k-values obtained before the movement.

30. The method of claim 29 wherein the amount of movement is not coherent with the spatial frequencies.

31. The method of claim 25 wherein the spatial frequencies are closely spaced.

32. The method of claim 25 further comprising obtaining k-values representing long wavelength spatial frequencies and normalizing the k-values to be compared with k-values from the same spatial frequencies and direction in bones with known degrees of disease before the comparison.

33. The method of claim 32 wherein the k-values from the same spatial frequencies and direction in bones with known degrees of disease are normalized using k-values representing the same long wavelength spatial frequencies for each bone with the respective known degree of disease.

34. The method of claim 25 wherein the k-values obtained are also compared with k-values at the same spatial frequencies and direction in the patient as previously obtained.

35. A computer-readable medium for use in assessing the health of trabecular bone, the computer-readable medium containing executable program instructions for:
controlling an magnetic resonance device to obtain k-values representing specific spatial frequencies and direction in the trabecular bone; and,
comparing those k-values with k-values from the same spatial frequencies and direction in bones with known degrees of disease.

36. A computer-readable medium for use in assessing the health of trabecular bone, the computer-readable medium containing executable program instructions for:
controlling an magnetic resonance device to obtain k-values representing specific spatial frequencies and direction in the trabecular bone; and,
comparing those k-values with previously obtained k-values at the same spatial frequencies and direction in the same bone.
